(19) United States
(12) Patent Application Publication
(10) Pub. No.: US 2020/0224142 A1
(43) Pub. Date: Jul. 16, 2020

(54) CELL CULTURE DEVICE

(71) Applicant: OLYMPUS CORPORATION, Tokyo (JP)

(72) Inventor: Takuma DEZAWA, Tokyo (JP)

(73) Assignee: OLYMPUS CORPORATION, Tokyo (JP)

(21) Appl. No.: 16/708,823

(22) Filed: Dec. 10, 2019

(30) Foreign Application Priority Data
Jan. 11, 2019 (JP) 2019-003019

(51) Int. Cl. C12M 1/12; C12M 1/06; C12M 1/34; C12M 1/00

(52) U.S. Cl. CPC C12M 25/02; C12M 27/02; C12M 23/38; C12M 29/00; C12M 41/32

(57) ABSTRACT

The frequency at which a culture medium is replaced is reduced by prolonging the service life of the culture medium, the size of a device is reduced, cost is reduced, and a mechanical strength is enhanced. Provided is a cell culture device including: a culture container capable of accommodating cells and a culture solution; and an adsorbing member that is formed of a porous material having adsorption holes, the hole diameters of which are smaller than the cells and larger than impurities in the culture solution, and that adsorbs the impurities, without adsorbing the cells, in the culture solution by means of the adsorption holes while at least a portion of the adsorbing member is immersed in the culture solution in the culture container.

### CROSS-REFERENCE TO RELATED APPLICATIONS

This application is based on Japanese Patent Application No. 2019-003019, the content of which is incorporated herein by reference.

### TECHNICAL FIELD

The present invention relates to cell culture devices.

### BACKGROUND ART

In recent years, there has been a growing demand for large-scale cell culture in the regenerative medical field in which cultured cells, such as induced pluripotent stem cells (iPS cells), are used. Unlike adherent culture in which a container referred to as a well plate or a dish is used, suspension culture in which a suspension culture container referred to as a bioreactor is used is characterized in that the culture efficiency is not affected by the cell adhesion area and is hence becoming mainstream in mass cell production.

Cell culture always involves deterioration of the culture medium and excretion of waste from cells. Suspension culture requires a culture medium preparation method appropriate for large-scale cell culture. For example, there are well-known methods for replacing a culture medium and for prolonging the service life of a culture medium (refer to, for example, PTLs 1 to 3). The culture medium preparation method described in PTL 1 is a culture-medium replacing method, which is carried out in the form of perfusion culture in which a new culture medium is supplied whereas waste liquid and the deteriorated culture medium are discharged. The culture medium preparation methods described in PTLs 2 and 3 are methods for prolonging the service life of a culture medium by removing waste in the culture medium and are carried out in the form of dialysis in which metabolic waste (small molecules) is removed whereas growth factors (polymers), such as proteins and cells, are preserved or in the form of conditioned culture in which the deteriorated culture medium is cleaned and thereafter returned to the culture container.

### CITATION LIST

### Patent Literature

{PTL 1}

Japanese Unexamined Patent Application, Publication No. 2015-136330
{PTL 2}

Japanese Unexamined Patent Application, Publication No. 2003-180334
{PTL 3}

Japanese Unexamined Patent Application, Publication No. 59-175877

### SUMMARY OF INVENTION

### Technical Problem

One aspect of the present invention is a cell culture device including: a culture container capable of accommodating cells and a culture medium; and an adsorbing member that is formed of a porous material having adsorption holes, the hole diameters of which are smaller than the cells and larger than impurities in the culture medium, and that adsorbs the impurities, without adsorbing the cells, by means of the adsorption holes while at least a portion of the adsorbing member is immersed in the culture medium in the culture container.

### BRIEF DESCRIPTION OF DRAWINGS

FIG. 1 is a schematic configuration diagram of a cell culture device according to a first embodiment of the present invention.

FIG. 2 is a magnified view of an adsorbing member, showing one example of adsorption holes of the adsorbing member.

FIG. 3 is a schematic configuration diagram of a cell culture device according to a first modification of the first embodiment of the present invention.

FIG. 4 is a schematic configuration diagram of a cell culture device according to a second modification of the first embodiment of the present invention.

FIG. 5 is a schematic configuration diagram of a cell culture device according to a third modification of the first embodiment of the present invention.

FIG. 6 is a schematic configuration diagram of a cell culture device according to a second embodiment of the present invention.

FIG. 7 is a schematic configuration diagram of a cell culture device according to a modification of the second embodiment of the present invention.

FIG. 8 is a schematic configuration diagram of a cell culture device according to a third embodiment of the present invention.

### DESCRIPTION OF EMBODIMENTS

### First Embodiment

A cell culture device according to a first embodiment of the present invention will now be described with reference to the drawings.

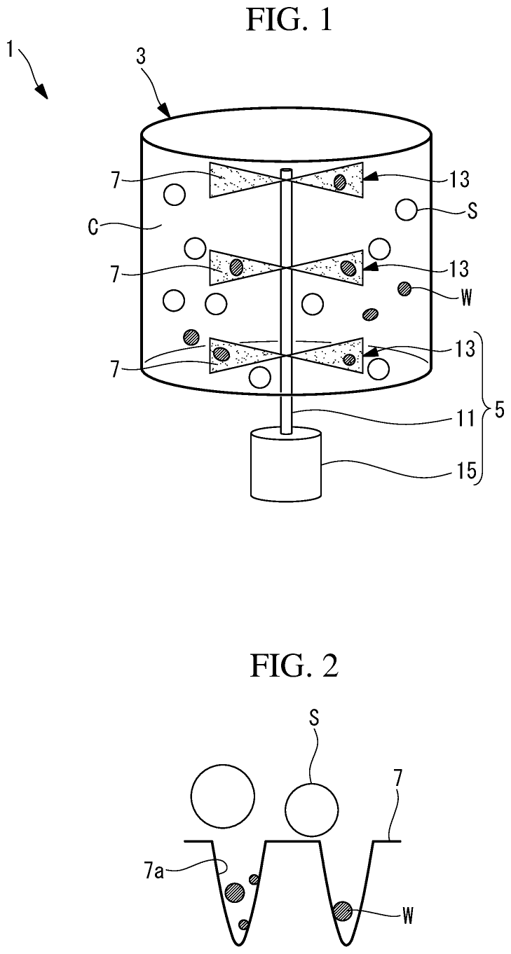

As shown in, for example, FIG. 1, a cell culture device 1 according to this embodiment includes: a culture container 3 capable of accommodating cells S and a culture solution (culture medium) C; a stirring mechanism 5 for stirring the culture solution C in the culture container 3; and adsorbing members 7 for adsorbing impurities in the culture solution C.

Examples of impurities include solids, such as fragments of a cell membrane, contained in the culture solution C, as well as cell-derived secretions, such as waste W excreted from the cells S. Examples of the waste W include lactic acid, ammonia, and carbon dioxide.

The culture container 3 is, for example, a bottomed cylindrical container, the top surface of which is closed.

The stirring mechanism 5 includes: a stirring shaft 11 that is inserted into the culture container 3 via the bottom of the culture container 3; a plurality of stirring vanes 13 provided on the stirring shaft 11; and a drive unit 15, such as a motor, for rotating the stirring shaft 11 about the longitudinal axis.

The plurality of stirring vanes 13 are provided, for example, at three longitudinal-direction positions of the stirring shaft 11 such that at least two stirring vanes 13 are arranged at each of the three longitudinal-direction positions in a manner spaced apart from each other in the circumferential direction of the stirring shaft 11. The plurality of stirring vanes 13 rotate about the stirring shaft 11 in the culture container 3 as a result of the stirring shaft 11 rotating about the longitudinal axis.

This stirring mechanism 5 can stir the culture solution C in the culture container 3 by rotating the stirring vanes 13 about the stirring shaft 11 by means of the drive unit 15. Also, the cells S can be cultured while being suspended in the culture solution C by causing the stirring mechanism 5 to stir the culture solution C.

As shown in, for example, FIG. 2, each of the adsorbing members 7 is formed of a porous material having many adsorption holes 7a, the hole diameters of which are smaller than the cells S and larger than impurities, such as the waste W, in the culture solution C and constitutes at least a portion of the corresponding stirring vane 13. Each of the adsorbing members 7 may constitute, for example, the corresponding stirring vane 13 itself or may cover the entire surface or a partial surface of the corresponding stirring vane 13.

For example, zeolite is used for the adsorbing members 7. Zeolite is polarized at the surfaces of adsorption holes thereof and is characterized by adsorbing polar molecules. When zeolite is employed for the adsorbing members 7, for example, ammonia, serving as the waste W, in the culture solution C can be adsorbed.

When zeolite is employed for the adsorbing members 7, ammonia, serving as the waste W, in the culture solution C can be selectively adsorbed. Zeolite has a capability of ion exchange and can take advantage of the capability of selectively adsorbing ammonia by replacing sodium ions Na+ belonging to zeolite with ammonium ions NH4+ in the culture medium. Note that ammonia in water is present in the form of ammonium ions.

Examples of the cells S to be cultured with the cell culture device 1 according to this embodiment include iPS cells of a human. The diameters of many iPS cells of a human fall within the range of, for example, 10-30 μm. Proteins in blood serum components, said proteins serving as a growth factor of the cells S, have a particle diameter of, for example, several nanometers to several tens of nanometers. The waste W, such as lactic acid, ammonia, and carbon dioxide, as well as glucose (specific substance) serving as an energy source for the activity of the cells S, has a particle diameter of, for example, less than 1 nm.

Each of the adsorbing members 7 in this embodiment includes the adsorption holes 7a in the form of micropores, the diameters of which are 2 nm or less. As a result of the adsorption holes 7a being formed as micropores, the diameters of which are 2 nm or less, the adsorbing member 7 while being immersed in the culture solution C in the culture container 3 can selectively adsorb the waste W in the culture solution C by preventing adsorption of not only the cells S but also proteins, serving as a growth factor required for growth of the cells S. It should be noted, however, that the adsorption holes 7a are larger than 0.38 nm, which is the molecular diameter of a water molecule.

The operation of the cell culture device 1 according to this embodiment will now be described.

When the cells S are to be cultured with the cell culture device 1 configured as described above, first, the cells S together with the culture solution C are accommodated in the culture container 3, and then the culture solution C in the culture container 3 is stirred by using the stirring mechanism 5. By doing so, the cells S can be cultured in the culture container 3 while being suspended in the culture solution C.

Here, when the stirring vanes 13 of the stirring mechanism 5 are rotated in the culture solution C, the culture solution C in the culture container 3 is stirred, and consequently, the adsorbing members 7, which constitute the stirring vanes 13, adsorb impurities, in the culture solution C, that are smaller than the hole diameters of the adsorption holes 7a while preventing the adsorption of the cells S, which are larger than the hole diameters of the adsorption holes 7a. In this case, the waste W excreted from the cells S is usually smaller than the cells S, and therefore, the waste W, which is an impurity, in the culture solution C can be adsorbed by the adsorbing members 7.

Therefore, according to the cell culture device 1 of this embodiment, the waste W from the cells S can be efficiently removed by the adsorbing members 7, thereby making it possible to prolong the service life of the culture solution C. In addition, the cell culture device 1 only requires a simple configuration in which the cells S are sorted from the waste W thereof in accordance with the hole diameters of the adsorption holes 7a of the adsorbing members 7, thus eliminating the need for inclusion of a dialysis unit, a waste-liquid treatment unit, a culture-medium replacing unit, and so forth even in the case where the cell culture device 1 is increased in scale. Furthermore, compared with a dialysis film, the adsorbing members 7, such as activated charcoal, are expected to be resistive against deformation and disruption. This makes it possible to achieve less frequent replacement of the culture medium, a compact size of the device, a reduction in cost, and enhancement in mechanical strength.

In addition, because each of the adsorbing members 7 constitutes at least a portion of the corresponding stirring vane 13, the adsorbing member 7 can be readily brought into contact with impurities in the entire culture solution C in the culture container 3 as the stirring vane 13 rotates. Therefore, the waste W can be highly efficiently adsorbed by the adsorbing members 7.

Although this embodiment has been described by way of an example of zeolite as the adsorbing members 7, instead of this, silica, alumina, activated charcoal, or the like may be employed. Activated charcoal is not polarized at the surfaces of the adsorption holes thereof and hence is characterized by adsorbing nonpolar molecules more readily than polar molecules. In addition, activated charcoal can adsorb lactic acid, serving as the waste W, in the culture solution C. It should be noted, however, that in the case where the pH indicates acidity, activated charcoal has an enhanced rate of adsorbing lactic acid and is characterized in that the lower the pH is, due to, for example, deterioration in the culture solution C, the higher the adsorbing force is.

This embodiment can be modified to have the following configurations.

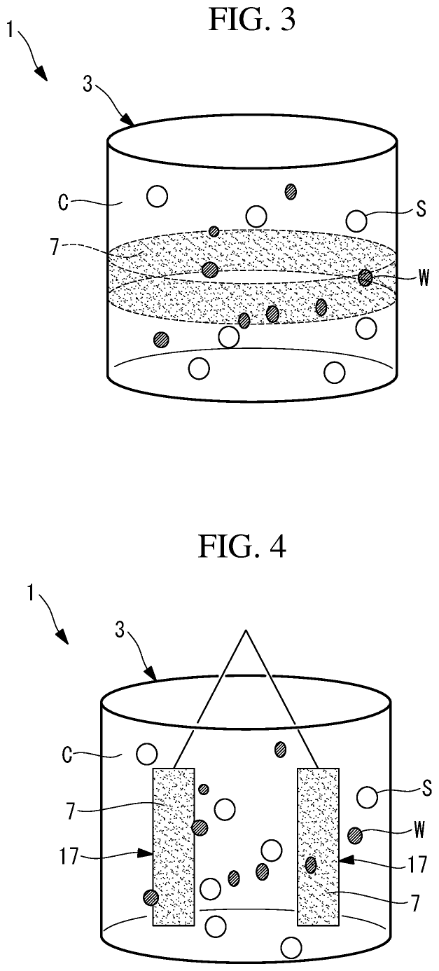

As shown in, for example, FIG. 3, in a first modification, an adsorbing member 7 may constitute at least a portion of the inner wall surface of the culture container 3. FIG. 3 omits graphic representation of the stirring mechanism 5 for the sake of convenience for explanation. Similarly, FIGS. 4 to 8 also omit graphic representation of the stirring mechanism 5.

According to this modification, the waste W that is present in the culture solution C and that comes into contact with the inner wall surface of the culture container 3 can be adsorbed by the adsorbing member 7. In this case, impurities in the entire culture solution C are readily brought into contact with the inner wall surface of the culture container 3 by causing the stirring mechanism 5 (refer to FIG. 1) to stir the culture solution C, thus making it possible to enhance the efficiency for adsorbing the waste W by means of the adsorbing member 7.

As shown in, for example, FIG. 4, in a second modification, the adsorbing member 7 may constitute at least a portion of an insertion member 17 that can be inserted into and removed from the culture container 3. The insertion member 17 has, for example, an elongated shape, and two of the insertion members 17 are inserted in the culture container 3.

Each of the adsorbing members 7 may constitute, for example, the corresponding insertion member 17 itself or may cover the entire surface or a partial surface of the corresponding insertion member 17.

According to this modification, the waste W in the culture solution C can be efficiently adsorbed by the adsorbing members 7 by immersing or removing the insertion members 17 into or from the culture solution C in the culture container 3 according to the necessity for removing the waste W in the culture solution C. Because the waste W increases in amount as a culture process proceeds, the waste W can be efficiently adsorbed by immersing the insertion members 17 in the culture solution C at a stage where a certain amount of the waste W is accumulated. More specifically, in a state where the concentration of the waste W is high, the waste W can be efficiently removed because substances necessary for the cells S are adsorbed with a relatively low probability in such a state. In addition, when the adsorbing members 7 become dirty or the adsorbing performance of the adsorbing members 7 deteriorates, the adsorbing members 7 can be disposed of together with the insertion members 17 as a whole. Although two insertion members 17 having an elongated shape are given as an example in FIG. 4, the number and the shape of the insertion members 17 are not limited to those described above.

In the second modification, the timing at which the adsorbing members 7 are immersed into or removed from the culture solution C may be determined on the basis of the concentration of the specific substance measured by a sensor 21. In this case, if the concentration of the specific substance in the culture solution C is lower than a predetermined threshold value, the adsorbing members 7 can be immersed in the culture solution C. By doing so, adsorption of the substance necessary for the cells S can be efficiently prevented.

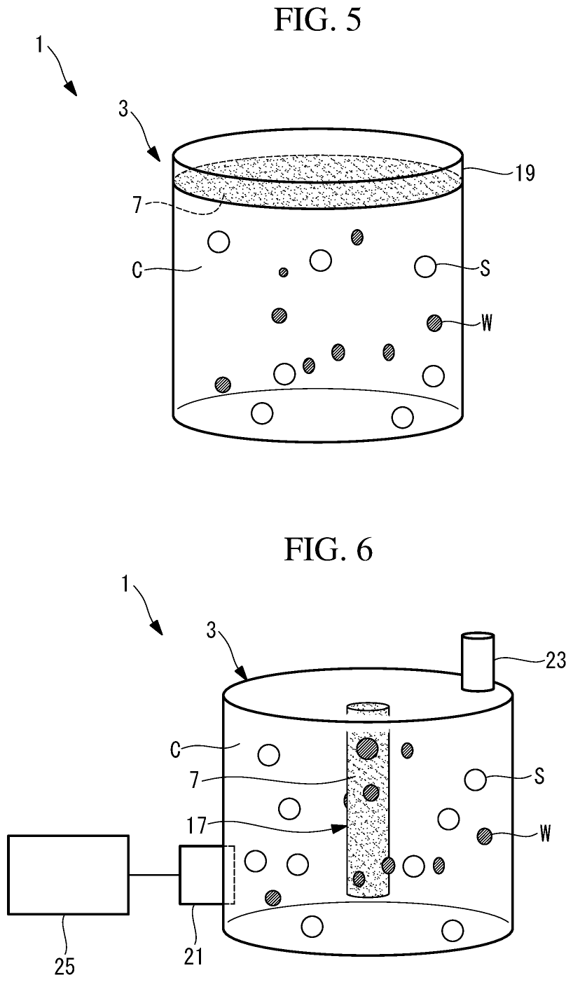

As shown in, for example, FIG. 5, in a third modification, an adsorbing member 7 may be provided on a lid section 19 for closing an opening of the culture container 3.

According to this modification, the waste W in the culture solution C can be adsorbed by the adsorbing member 7 as a result of the culture solution C coming into contact with the inner surface of the lid section 19 in a state where the opening of the culture container 3 is closed by the lid section 19.

### Second Embodiment

Next, a cell culture device according to a second embodiment of the present invention will be described.

As shown in FIG. 6, a cell culture device 1 according to this embodiment differs from the cell culture device according to the first embodiment in that the cell culture device 1 according to this embodiment includes a sensor (concentration-measuring unit) 21 for monitoring nutrient molecules (specific substance) in the culture solution C and a supply unit (specific-substance supply unit) 23 for supplying nutrient molecules to the culture solution C in the culture container 3.

The same components in this embodiment as those used in the cell culture device 1 according to the first embodiment are denoted by the same reference signs, and thus descriptions thereof will be omitted.

The sensor 21 is disposed in the culture container 3 and measures the concentration of the specific substance contained in the culture solution C in the culture container 3. Examples of the specific substance include glucose. Although glucose is described as an example of the specific substance in the following explanation, the specific substance is not limited to glucose but may be, for example, a particular amino acid or fatty acid.

The supply unit 23 supplies glucose to the culture solution C in the culture container 3.

In this embodiment, the adsorbing member 7 constitutes, for example, at least a portion of the insertion member 17 that can be inserted into and removed from the culture container 3.

According to the cell culture device 1 of this embodiment, a user can know the amount of glucose in the culture solution C by means of the sensor 21. Also, when the amount of glucose contained in the culture solution C in the culture container 3 becomes insufficient, the culture solution C can be replenished with glucose by means of the supply unit 23.

As shown in, for example, FIG. 6, in this embodiment, a display unit 25 for displaying the concentration of glucose measured by the sensor 21 may be provided. With the display unit 25, the user can easily know the amount of glucose in the culture solution C.

In addition, this embodiment may be configured so that when the concentration measured by the sensor 21 is equal to or smaller than a predetermined threshold value, the supply unit 23 may automatically replenish the culture solution C with glucose. In this case, the supply unit 23 can be controlled by a control unit (not shown in the figure) including, for example, a storage unit such as a hard disk drive, a CPU, and a RAM.

More specifically, a supply program executed by the CPU is stored in the storage unit, the CPU reads the supply program stored in the storage unit, it is determined whether or not the concentration measured by the sensor 21 is equal to or smaller than the predetermined threshold value, and when it is determined that the concentration measured by the sensor 21 is equal to or smaller than the predetermined threshold value, glucose is supplied from the supply unit 23.

In this embodiment, for example, a dialysis film may be employed, instead of employing the sensor 21. In this case, the dialysis film may be provided at the site in the culture container 3 to which glucose is supplied from the supply unit 23, so that when the concentration of glucose in the culture solution C decreases, glucose automatically passes through the dialysis film due to a concentration gradient and is supplied into the culture solution C.

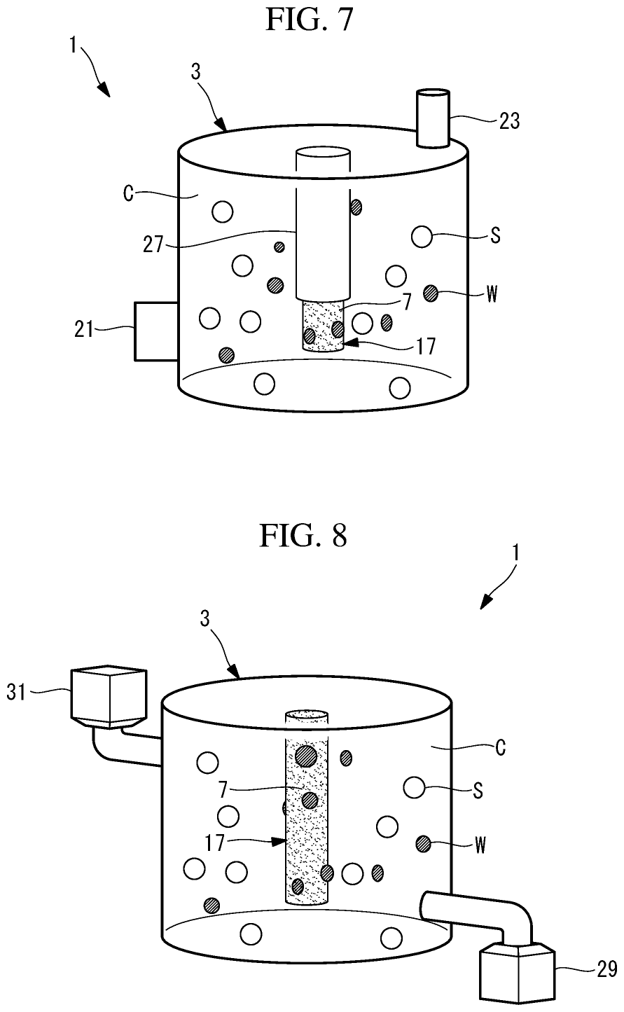

As shown in, for example, FIG. 7, this embodiment may include a covering member 27 that covers the adsorbing member 7 so as to be attachable to and detachable from the adsorbing member 7. It is preferable that the covering member 27 have, for example, a cylindrical shape so as to be capable of accommodating the insertion member 17 and prevent the adsorption holes 7a of the adsorbing member 7 from being exposed while the covering member 27 covers the adsorbing member 7. FIG. 7 shows a state where a portion of the adsorbing member 7 is exposed from the covering member 27.

In this case, if the adsorbing member 7 is immersed in the culture solution C with the adsorption holes 7a exposed, for example, from the initial stage at the beginning of culture, there is risk that a nutritional substance in the culture solution C is adsorbed as an impurity by the adsorbing member 7 before the cells S excrete the waste W. In contrast, adsorption of such a nutritional substance in the culture solution C can be suppressed by leaving the adsorbing member 7 covered with the covering member 27 immersed in the culture solution C in the culture container 3, in which case the adsorption holes 7a are exposed by detaching the covering member 27 from the adsorbing member 7 according to the necessity for removing the waste W in the culture solution C.

The covering member 27 may be formed of a water-soluble material. Examples of the water-soluble material include starch. While the adsorbing member 7 that is covered with the covering member 27 formed of a water-soluble material is immersed in the culture solution C in the culture container 3, the covering member 27 is dissolved in the culture solution C over time, making it possible to adjust the timing at which the adsorption holes 7a are exposed.

This embodiment may be configured so that when the concentration of glucose measured by the sensor 21 is equal to or smaller than the predetermined threshold value, the covering member 27 is actuated to expose the adsorption holes 7a in the culture solution C. In this case, the covering member 27 may have, for example, a window section that opens on the basis of a signal from the sensor 21, said signal indicating that the concentration of glucose is equal to or smaller than the predetermined threshold value. Also, the adsorption holes 7a may be exposed as a result of the window section of the covering member 27 being opened.

By doing so, it is possible to prevent a nutritional substance in the culture solution C from being adsorbed as an impurity by the adsorbing member 7. In addition, by controlling the timing at which the member 27 is attached and detached in accordance with the concentration of glucose measured by the sensor 21, it is possible to quantify the timing at which the covering member 27 is attached and detached without depending on the subjective sense of a person carrying out culturing (user).

### Third Embodiment

Next, a cell culture device according to a third embodiment of the present invention will be described.

As shown in FIG. 8, a cell culture device 1 according to this embodiment differs from the cell culture device according to the first embodiment in that the cell culture device 1 according to this embodiment includes a culture-medium discharge unit 29 for discharging the culture solution C in the culture container 3 and a culture-medium supply unit 31 for supplying the culture solution C into the culture container 3.

The same components in this embodiment as those used in the cell culture device 1 according to the first embodiment are denoted by the same reference signs, and thus descriptions thereof will be omitted.

The culture-medium discharge unit 29 and the culture-medium supply unit 31 are both connected to the culture container 3.

In the culture container 3, a transmission section (not shown in the figure) through which impurities can pass and the cells S cannot pass is provided between the culture-medium discharge unit 29 and the culture-medium supply unit 31. Examples of the transmission section include a dialysis film.

According to the cell culture device 1 of this embodiment, the culture solution C in the culture container 3 can be replaced by discharging the old culture solution C in the culture container 3 by means of the culture-medium discharge unit 29 and supplying a new culture solution C into the culture container 3 by means of the culture-medium supply unit 31. In this case, when the culture solution C is replaced, the transmission section can prevent the cells S from being discharged together with the culture solution C from the culture container 3. The discharged culture solution C may be recycled.

Although the embodiments of the present invention have been described in detail with reference to the drawings, the specific structure is not limited to those of these embodiments but includes design changes etc. that do not depart from the spirit of the present invention. The present invention is not limited to the invention applied to each of the above-described embodiments and modifications but can be applied to, for example, embodiments in which these embodiments and modifications are appropriately combined and is not particularly limited. In addition, although each of the above-described embodiments has been described by way of an example of a configuration including the stirring mechanism 5, the adsorbing member 7 may be applied to a configuration that does not include the stirring mechanism 5.

Although the above-described embodiments have been described by way of an example of iPS cells of a human as the cells S to be cultured, any other cells that can be suspension-cultured can be used as the cells S to be cultured. For example, the cells S to be cultured may be hematopoietic cells such as red blood cells, blood platelets, and lymphocytes. For example, cells may be suspension-cultured by adhering adherent cells to particulate base materials. The diameters of holes in the adsorbing member may be optimized according to the cells S to be cultured.

The above-described embodiments have been described by way of an example where the adsorbing member is realized by at least a portion of each of the stirring vane, the lid section, the inner wall surface of the culture container, and the insertion member. However, the stirring vane including the adsorbing member, the lid section including the adsorbing member, the culture container including the adsorbing member, and the insertion member including the adsorbing member may be configured to be disposable. Although the culture container having an inner wall surface serving as the adsorbing member is employed, the inner wall surface may be configured to be insertable and removable so that the inner wall surface can be disposed of, and furthermore, the culture container itself may be configured to be disposable. The timing at which the members are to be replaced can be determined by the user. More specifically, a member can be replaced, for example, when the culture medium is replaced, when culture is finished, when the adsorption efficiency of the member is decreased due to deterioration in the member, and so forth. All members may be configured to be disposable so that all members are replaced as a whole when replaced, or alternatively, only some members may be configured to be disposable so that only applicable members are replaced as appropriate. It is preferable that members be prepared and stored in a covered state to prevent unwanted molecules from coming into contact with the adsorbing members at the time of replacement. By doing so, the user can prevent a decrease in the adsorption efficiency due to prolonged use of the adsorbing members and contamination during culture. This leads to efficient culture.

According to the present invention, it is possible to provide a stirring vane for stirring the cells and culture medium that are contained in the culture container capable of accommodating cells and a culture medium, wherein at least a portion of the stirring vane is formed of a porous material having adsorption holes the hole diameters of which are smaller than the cells and larger than impurities in the culture medium, thus allowing the stirring vane to adsorb the impurities while preventing the cells from being adsorbed by the adsorption holes. The waste can be efficiently removed from the culture system by replacing this stirring vane, as appropriate, at a proper timing during cell culture.

Consequently, the following aspects are derived from the above-described embodiments.

One aspect of the present invention is a cell culture device including: a culture container capable of accommodating cells and a culture medium; and an adsorbing member that is formed of a porous material having adsorption holes, the hole diameters of which are smaller than the cells and larger than impurities in the culture medium, and that adsorbs the impurities, without adsorbing the cells, by means of the adsorption holes while at least a portion of the adsorbing member is immersed in the culture medium in the culture container.

According to the cell culture device of this aspect, the culture medium and the cells are accommodated in the culture container, and then at least a portion of the adsorbing member is immersed in the culture medium in the culture container. This causes the adsorbing member to adsorb impurities in the culture medium because the impurities are smaller than the hole diameters of the adsorption holes, while preventing the adsorbing member from adsorbing the cells, which are larger than the hole diameters of the adsorption holes.

In this case, waste excreted from the cells is normally smaller than the cells and hence is adsorbed by the adsorbing member as impurities in the culture medium. Therefore, the waste from the cells can be efficiently removed by the adsorbing member, thus making it possible to prolong the service life of the culture medium. In addition, the cell culture device only requires a simple configuration in which the cells are sorted from the waste thereof in accordance with the hole diameters of the adsorption holes of the adsorbing member, thus eliminating the need for including a dialysis unit, a waste-liquid treatment unit, a culture-medium replacing unit, and so forth even in the case where the cell culture device is increased in scale. Furthermore, compared with a dialysis film, the adsorbing member is expected to be resistive against deformation and disruption. This makes it possible to achieve less frequent replacement of the culture medium, a compact size of the device, a reduction in cost, and enhancement in mechanical strength.

In the cell culture device according to the above-described aspect, the hole diameters of the adsorption holes may be smaller than proteins contained in the culture medium.

With this configuration, it is possible to prevent proteins, which are substances necessary for the growth of the cells contained in the culture medium, from being adsorbed by the adsorbing member as impurities in the culture medium.

In the cell culture device according to the above-described aspect, the adsorbing member may be activated charcoal, silica, alumina, or zeolite.

With this configuration, a normally available substance can be used as the porous material, leading to a further reduction in cost.

The cell culture device according to the above-described aspect may further include a stirring mechanism having a stirring vane that is disposed in the culture container and that is rotatable about a predetermined rotating shaft.

With this configuration, the culture medium in the culture container can be stirred by rotating, about the predetermined rotating shaft, the stirring vane of the stirring mechanism in the culture container. Therefore, with a simple configuration in which the culture medium is stirred by the stirring mechanism, the cells can be cultured while being suspended in the culture medium in the culture container.

In the cell culture device according to the above-described aspect, the adsorbing member may constitute at least a portion of the stirring vane.

With this configuration, when the stirring vane of the stirring mechanism is rotated, the impurities in the culture medium can be adsorbed by the adsorbing member while the culture medium in the culture container is being stirred. In this case, the adsorbing member can be readily brought into contact with the impurities in the entire culture medium in the culture container as the stirring vane rotates. Therefore, the impurities can be highly efficiently adsorbed.

In the cell culture device according to the above-described aspect, the adsorbing member may constitute at least a portion of an inner wall surface of the culture container.

With this configuration, the impurities in the culture medium, said impurities being in contact with the inner wall surface of the culture container, can be adsorbed by the adsorbing member. In this case, impurities in the entire culture medium are readily brought into contact with the inner wall surface of the culture container by causing the stirring mechanism to stir the culture medium, thus making it possible to enhance the efficiency for adsorbing the impurities by means of the adsorbing member.

In the cell culture device according to the above-described aspect, the adsorbing member may constitute at least a portion of an insertion member that can be inserted into and removed from the culture container.

With this configuration, the impurities in the culture medium can be efficiently adsorbed by the adsorbing member by inserting or removing the insertion member into or from the culture container according to the necessity for removing the waste in the culture medium.

The cell culture device according to the above-described aspect may further include a lid section for closing an opening of the culture container, wherein the adsorbing member may be provided on an inner surface of the lid section.

With this configuration, the impurities in the culture medium are adsorbed by the adsorbing member as a result of the culture medium coming into contact with the inner surface of the lid section in a state where the opening of the culture container is closed by the lid section.

The cell culture device according to the above-described aspect may further include a concentration-measuring unit that is disposed in the culture container and that measures a concentration of a specific substance contained in the culture medium in the culture container.

With this configuration, the amount of the specific substance in the culture medium can be known by using the concentration-measuring unit. Examples of the specific substance include glucose, which is an energy source for the activity of the cells.

The cell culture device according to the above-described aspect may further include a display unit for displaying the concentration of the specific substance, the concentration being measured by the concentration-measuring unit.

With this configuration, the amount of the specific substance in the culture medium can be easily known by checking the display unit.

The cell culture device according to the above-described aspect may further include a specific-substance supply unit for supplying the specific substance to the culture medium in the culture container.

With this configuration, when the amount of the specific substance contained in the culture medium in the culture container becomes insufficient, the specific-substance supply unit can replenish the culture medium with the specific substance.

In the cell culture device according to the above-described aspect, when the concentration measured by the concentration-measuring unit is equal to or smaller than a predetermined threshold value, the specific-substance supply unit may supply the specific substance to the culture medium.

With this configuration, the specific-substance supply unit can efficiently replenish the culture medium with the specific substance in accordance with the amount of the specific substance in the culture medium in the culture container.

The cell culture device according to the above-described aspect may further include a covering member that can cover the adsorbing member and that can be attached to and detached from the adsorbing member.

With this configuration, the adsorption holes of the adsorbing member are not exposed while the adsorbing member is covered with the covering member, preventing adsorption of the impurities in the culture medium. In this case, if the adsorbing member is immersed in the culture medium in a state where the adsorption holes are exposed, for example, from the initial stage at the beginning of culture, there is a risk of a nutritional substance in the culture medium being adsorbed as an impurity by the adsorbing member before the cells excrete waste. In contrast, adsorption of such a nutritional substance in the culture medium can be suppressed by adjusting the timing at which the adsorption holes of the adsorbing member are exposed by means of the covering member according to the necessity for removing the waste in the culture medium while the adsorbing member is left immersed in the culture medium in the culture container.

In the cell culture device according to the above-described aspect, the covering member may be a water-soluble material.

With this configuration, while the adsorbing member covered with the covering member is immersed in the culture medium in the culture container, the covering member dissolves in the culture medium over time, making it possible to adjust the timing at which the adsorption holes of the adsorbing member are exposed.

In the cell culture device according to the above-described aspect, when the concentration of the specific substance contained in the culture medium, the concentration being measured by the concentration-measuring unit, is lower than a predetermined threshold value, the adsorbing member may come into contact with the culture medium.

With this configuration, it is possible to prevent the nutritional substance in the culture medium from being adsorbed as an impurity by the adsorbing member until the concentration of the specific substance contained in the culture medium becomes lower than the predetermined threshold value. Once the concentration of the specific substance contained in the culture medium becomes lower than the predetermined threshold value, the waste from the cells is removed by the adsorbing member as a result of the adsorbing member coming into contact with the culture medium, thereby making it possible to prolong the service life of the culture medium.

The cell culture device according to the above-described aspect may further include a culture-medium discharge unit that is connected to the culture container and that discharges the culture medium in the culture container; and a culture-medium supply unit that is connected to the culture container and that supplies the culture medium into the culture container.

With this configuration, the culture medium in the culture container can be replaced by discharging the old culture medium in the culture container by means of the culture-medium discharge unit and supplying a new culture medium into the culture container by means of the culture-medium supply unit.

The cell culture device according to the above-described aspect may further include a transmission section that is disposed between the culture-medium discharge unit and the culture-medium supply unit in the culture container and through which the impurities can pass and the cells cannot pass from a side of the culture-medium supply unit towards a side of the culture-medium discharge unit.

With this configuration, when the culture medium in the culture container is replaced, it is possible to prevent the transmission section from discharging the cells from the culture container together with the culture medium.

In the cell culture device according to the above-described aspect, the adsorbing member may adsorb waste excreted from the cells.

It is possible to prolong the service life of the culture medium by removing the waste from the cells by means of the adsorbing member.

The present invention affords an advantage in that the frequency at which the culture medium is replaced can be reduced by prolonging the service life of the culture medium, the size of the device can be reduced, cost can be reduced, and the mechanical strength can be enhanced.

### REFERENCE SIGNS LIST

1 Cell culture device
3 Culture container
5 Stirring mechanism
7 Absorbing member
7a Absorption hole
13 Stirring vane
17 Insertion member
19 Lid section
21 Sensor (concentration-measuring unit)
23 Supply unit
25 Display unit
27 Covering member
29 Culture-medium discharge unit
31 Culture-medium supply unit
C Culture solution (culture medium)
S Cell
W Waste

1. A cell culture device comprising:
a culture container capable of accommodating cells and a culture medium; and
an adsorbing member that is formed of a porous material having adsorption holes, the hole diameters of which are smaller than the cells and larger than impurities in the culture medium, and that adsorbs the impurities, without adsorbing the cells, by means of the adsorption holes while at least a portion of the adsorbing member is immersed in the culture medium in the culture container.

2. The cell culture device according to claim 1, wherein the hole diameters of the adsorption holes are smaller than proteins contained in the culture medium.

3. The cell culture device according to claim 1, wherein the adsorbing member is activated charcoal, silica, alumina, or zeolite.

4. The cell culture device according to claim 1, further comprising: a stirring mechanism having a stirring vane that is disposed in the culture container and that is rotatable about a predetermined rotating shaft.

5. The cell culture device according to claim 4, wherein the adsorbing member constitutes at least a portion of the stirring vane.

6. The cell culture device according to claim 4, wherein the adsorbing member constitutes at least a portion of an inner wall surface of the culture container.

7. The cell culture device according to claim 4, wherein the adsorbing member constitutes at least a portion of an insertion member that can be inserted into and removed from the culture container.

8. The cell culture device according to claim 1, further comprising:
a lid section for closing an opening of the culture container,
wherein the adsorbing member is provided on an inner surface of the lid section.

9. The cell culture device according to claim 1, further comprising: a concentration-measuring unit that is disposed in the culture container and that measures a concentration of a specific substance contained in the culture medium in the culture container.

10. The cell culture device according to claim 9, further comprising: a display unit for displaying the concentration of the specific substance, the concentration being measured by the concentration-measuring unit.

11. The cell culture device according to claim 9, further comprising: a specific-substance supply unit for supplying the specific substance to the culture medium in the culture container.

12. The cell culture device according to claim 11, wherein when the concentration measured by the concentration-measuring unit is equal to or smaller than a predetermined threshold value, the specific-substance supply unit supplies the specific substance to the culture medium.

13. The cell culture device according to claim 1, further comprising: a covering member that can cover the adsorbing member and that can be attached to and detached from the adsorbing member.

14. The cell culture device according to claim 13, wherein the covering member is a water-soluble material.

15. The cell culture device according to claim 9, wherein when the concentration of the specific substance contained in the culture medium, the concentration being measured by the concentration-measuring unit, is lower than a predetermined threshold value, the adsorbing member comes into contact with the culture medium.

16. The cell culture device according to claim 1, further comprising:
a culture-medium discharge unit that is connected to the culture container and that discharges the culture medium in the culture container; and
a culture-medium supply unit that is connected to the culture container and that supplies the culture medium into the culture container.

17. The cell culture device according to claim 16, further comprising: a transmission section that is disposed between the culture-medium discharge unit and the culture-medium supply unit in the culture container and through which the impurities can pass and the cells cannot pass from a side of the culture-medium supply unit towards a side of the culture-medium discharge unit.

18. The cell culture device according to claim 1, wherein the adsorbing member adsorbs waste excreted from the cells.

19. The cell culture device according to claim 5, wherein the stirring vane is configured to be disposable.

20. The cell culture device according to claim 6, wherein the culture container is configured to be disposable.
